(19) United States
(12) Patent Application Publication
(10) Pub. No.: US 2006/0089622 A1
(43) Pub. Date: Apr. 27, 2006

(54) Inserted molded switch cover for surgical instrument

(75) Inventors: John Bourne, Torrance, CA (US); Kenard W. Griffith, Carpinteria, CA (US); Douglas Blaine Francis, Ventura, CA (US)

Correspondence Address: HAYNES AND BOONE, LLP, 901 MAIN ST, SUITE 3100, DALLAS, TX, 75202, US

(73) Assignee: Medtronic, Inc., Minneapolis, MN (US)

(21) Appl. No.: 10/970,498

(22) Filed: Oct. 21, 2004

(51) Int. Cl. A61B 17/00

(52) U.S. Cl. 606/1

(57) ABSTRACT

A surgical instrument includes an outer casing having a first portion for surrounding a motor, a second portion having for housing a plurality of buttons, and a third portion for providing a small body by which a user can control and manipulate the surgical instrument. A plurality of buttons are provided in the second portion of the outer casing, the buttons for controlling an operation of the motor. A switch cover for engaging with the second portion of the outer casing is also provided. The switch cover includes a relatively stiff skeleton and a plurality of relatively flexible portions that correspond with the plurality of buttons when the switch cover is engaged with the second portion of the outer casing. The flexible portions are adapted to provide external control of the buttons while the switch cover is engaged.

### FIELD OF THE INVENTION

The present invention relates generally to surgical instruments and in particular to switch covers for surgical instruments.

### BACKGROUND

Many conventional surgical instruments employ motors to rotate or otherwise move a tool, such as a cutting element for the dissection of bone or other tissue. The motor usually includes a rotary shaft and the tool is coupled to the shaft and is moved by the motor.

It can be appreciated that these instruments can be relatively small yet the motor is required to drive the tool at relatively high speeds or torque. Furthermore, the environment that surgical instruments must operate in is quite unique. For example, instruments must not only maintain sterility during the surgical procedure, but must be sterilizable from one procedure to the next. This is often achieved through a high-temperature process known as autoclave, although other processes can be used.

Another unique aspect of the surgical environment is that it often includes excessive amounts of liquids (e.g., blood) that can be in constant contact with the instrument. The instrument must be sufficiently sealed to prevent any material from entering the instrument. Also, no part of the instrument can dislodge during the operation. Furthermore, a high level of precise control is often required for a successful operation.

U.S. Pat. No. 6,126,670, which is hereby incorporated by reference, describes a cordless surgical handpiece with disposable battery. The cordless surgical handpiece has a manually operated external trigger for activating and controlling the motor operations. It is desired to provide improvements and enhancements to the disclosed cordless surgical handpiece as well as other surgical instruments.

### SUMMARY

The present invention provides a mechanism for controlling a motor in a surgical instrument, such as a battery powered instrument, that is adapted to work in a surgical environment.

Various embodiments of the invention discussed below may possess one or more of the above features and advantages, or provide one or more solutions to the above problems existing in the prior art.

### BRIEF DESCRIPTION OF THE DRAWINGS

FIG. 1 illustrates a human patient for receiving a surgical procedure associated with one or more embodiments of the present invention.

FIG. 2 is an isometric view of a surgical instrument according to an embodiment of the present invention.

FIGS. 3a and 3b are an enlarged, partial sectional view of a skeleton section of a molded switch cover for use with the surgical instrument of FIGS. 1 and 2.

FIG. 4 is an exploded view of one embodiment of a molded switch cover for use with the surgical instrument of FIGS. 1 and 2.

FIGS. 5a and 5b are assembled views of the molded switch cover of FIG. 4.

FIGS. 6a and 6b are exploded views of the molded switch cover of FIGS. 5a and 5b with the surgical instrument of FIGS. 1 and 2. FIG. 6a includes a cut-away view of the molded switch cover of FIGS. 5a and 5b.

### DETAILED DESCRIPTION OF THE PREFERRED EMBODIMENTS

The present invention provides an improved surgical instrument, an improved switch mechanism, and an improved cover for the switch mechanism, and methods of assembly and using each.

For the purposes of promoting an understanding of the principles of the invention, references will now be made to the embodiments, or examples, illustrated in the drawings, and specific languages will be used to describe the same. It will nevertheless be understood that discussions of one or more specific examples and repetitions of one or more reference numerals is provided for the sake of clarity, and should not limit the scope of the invention. Any alterations and further modifications in the described embodiments, and any further applications of the principles of the invention as described herein are contemplated as would normally occur to one skilled in the art to which the invention relates.

Referring now to FIG. 1, the reference numeral 10 designates a surgical instrument for use on a human anatomy. It will become apparent to those skilled in the art that the described surgical instrument 10 is not limited to any particular surgical operation but has utility for various applications in which it is desired to dissect bone or other tissue. Applications include, but are not limited to:
1. Neurosurgery—Cranial, Spine, and Otology
2. Arthroscopy—Orthopedic
3. Endoscopic—Gastroenterology, Urology, Soft Tissue
4. Small Bone—Orthopedic, Oral-Maxiofacial, Ortho-Spine, and Otology
5. Cardio Thoracic—Small Bone Sub-Segment
6. Large Bone—Total Joint and Trauma
7. Dental.

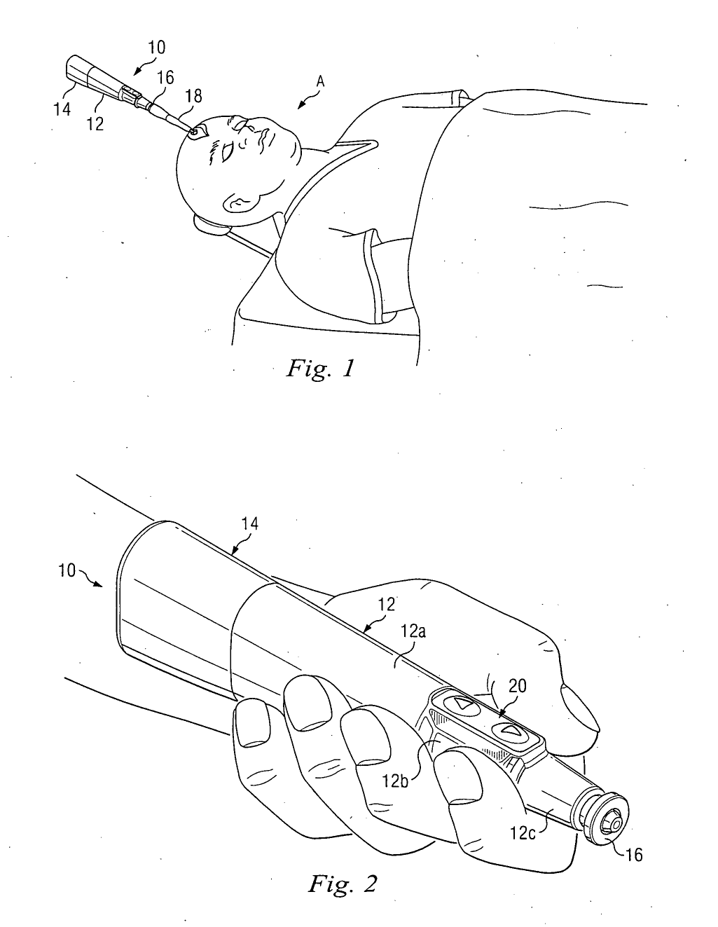

Referring to FIGS. 1 and 2 of the drawings, the surgical instrument 10, according to one embodiment of the invention, includes an outer casing 12 connected to a battery pack 14. The outer casing 12 encloses a motor for rotating a shaft connected to a coupler 16, which may further connect to a tool 18.

The outer casing 12 includes a gripping portion 12a which is sized and adapted to fit comfortably in a user's hand for allowing the user to hold and manipulate the surgical instrument 10. The gripping portion 12a is also of sufficient size and shape to house the motor and connect to the battery pack 14. It is understood that other embodiments may not use the battery pack 14, and may include such things as a hydraulic hose, an electrical power bus, or a rotating cable connected to an external motor.

The outer casing 12 also includes a switch portion 12b adjacent the gripping portion 12a. The switch portion 12b is recessed, as compared to the gripping portion 12a. The switch portion 12b includes a switch mechanism 20 that will be discussed in greater detail below. The switch mechanism 20 is configured and positioned so that it can be conveniently controlled by the user and can maintain the requirements of a surgical instrument (e.g., able to hold out moisture, withstand vibrations, and survive autoclave).

The outer casing 12 further includes a control portion 12c adjacent the switch portion 12b. The control portion 12c is recessed, as compared to the switch portion 12b, so that the three portions 12a, 12b, 12c “stair-step” down to reduced diameters. The control portion 12c is sized so that it can fit firmly against the user's finger tips, allowing precise control of the instrument 10.

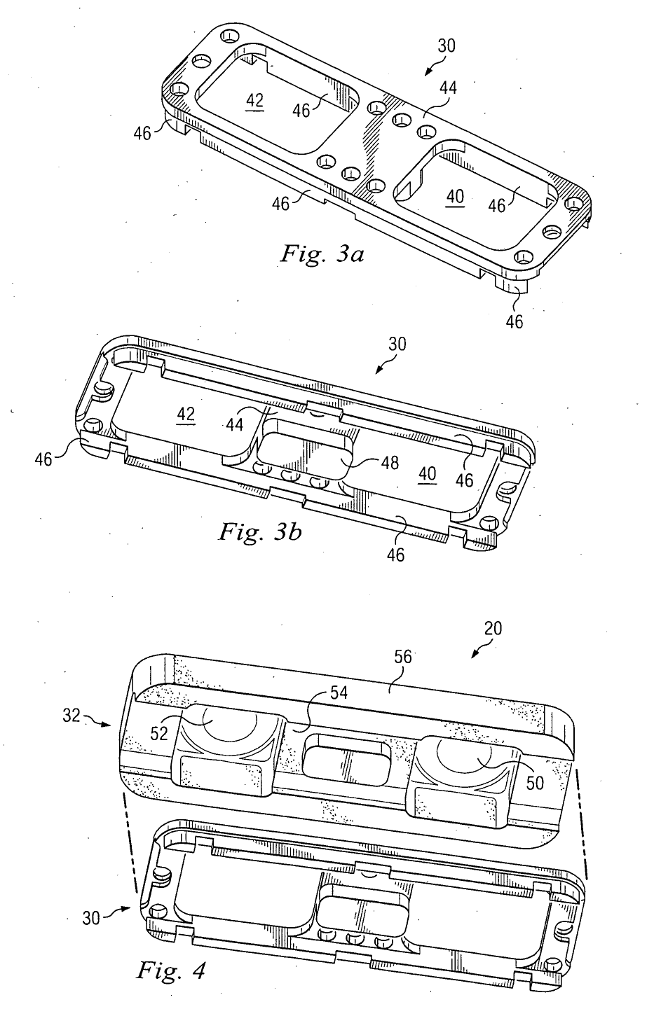

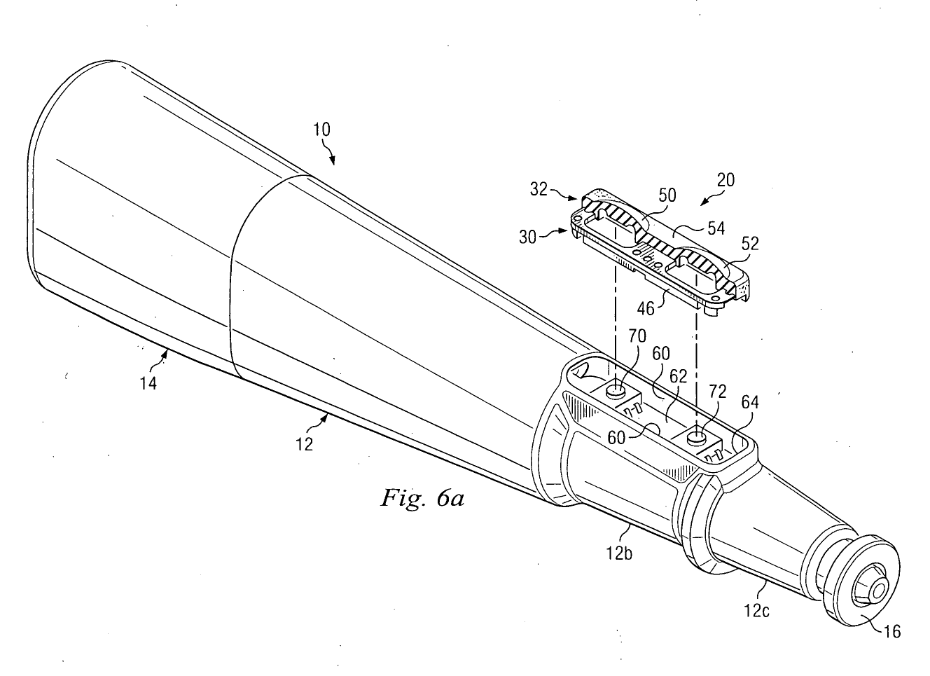

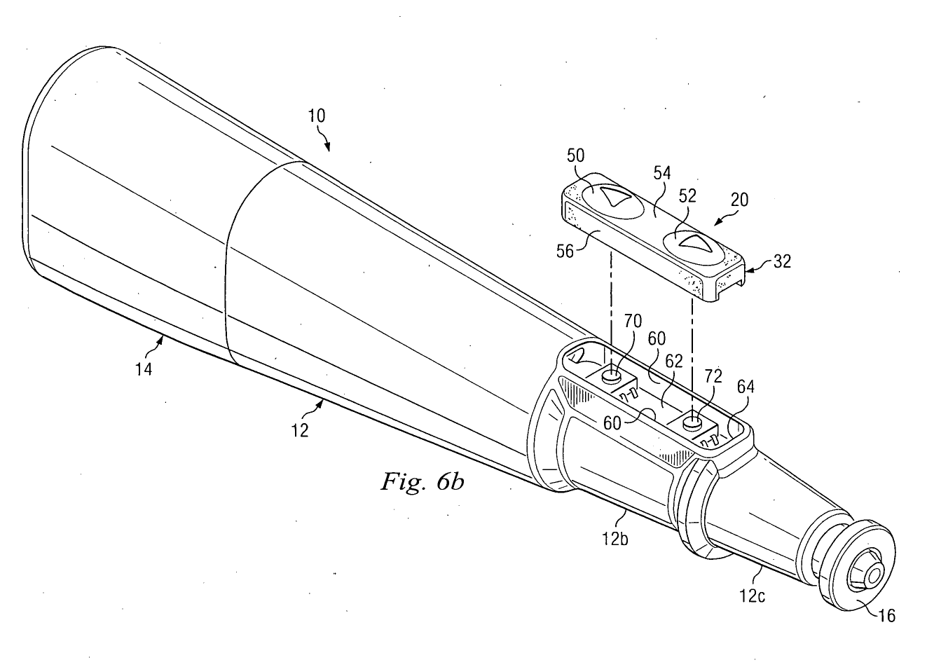

Referring to FIGS. 3a, 3b, 4, 5a, and 5b, the switch mechanism 20 includes two pieces—a rigid skeleton 30 and a flexible cover 32. Referring specifically to FIGS. 3a and 3b, the rigid skeleton 30 is made of an injection molded plastic. It includes two apertures 40, 42, a face 44, a plurality of extension members 46, and a spacer 48. The apertures 40, 42 are used to fit around electrical or mechanical buttons on the instrument 10, discussed in greater detail with respect to FIG. 6. The face 44 provides a solid, rigid structure for the entire switch mechanism 20. The face 44 includes a plurality of apertures for use in the injection molding process, and for interacting with the flexible cover 32. The extension members 46 are used to maintain the face 44 at a predetermined distance from the outside surface of the switch portion 12b of the outer casing 12 and for securing the switch mechanism 20 to the outer casing.

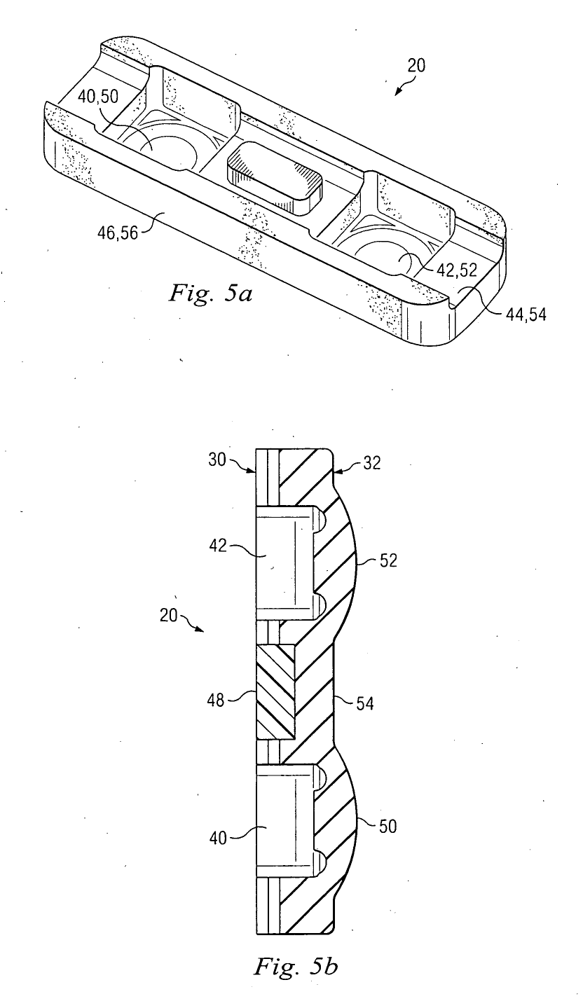

Referring to FIG. 4, the skeleton 30 is combined with the flexible cover 32 to form the switch mechanism 20. This can be done, for example, by first forming the rigid skeleton 30 and then heat-shrinking the flexible cover 32 thereon. The flexible cover 32 may be a silicon-based material that is safe for the surgical environment and is capable of permanently adhering to the skeleton 30. To be safe for the surgical environment, the flexible cover 32 must have a composition that will not disintegrate or otherwise react or interact with other items, both during surgery and during autoclave.

The flexible cover 32 includes two flexible button portions 50, 52 that correspond and align with the apertures 40, 42, respectively, of the skeleton 30. The flexible button portions 50, 52 are of a sufficient flexibility and size so that a user can operate underlying buttons on the surgical instrument 10, as discussed in greater detail below. The flexible cover 32 also includes a face portion 54 and side portions 56 that correspond with the face 44 and extension members 46, respectively, of the skeleton 20.

Referring now to FIGS. 6a and 6b, the switch mechanism 20 engages with the switch portion 12b of the outer casing 12. In one embodiment, the side portions 56 of the switch mechanism 20 frictionally engage with indented side portions 60 of the casing 12. The indented side portions 60 are configured to produce a recessed area 62 inside the switch portion 12b. One or more edges 64 in the recessed area 62 can be modified, such as being shaped with protrusions or a taper, to further fit and secure with corresponding indentions 66 in the switch mechanism 20.

In another embodiment, the side portions 56 of the switch mechanism 20 are lipped or flared so that they mechanically engage with the recessed area 62. In this embodiment, the switch mechanism 20 can be secured very tightly, thereby preventing it from becoming separated from the surgical instrument 10 during any surgical procedure.

The apertures 40, 42 of the switch mechanism 20 correspond with buttons 70, 72, respectively. It is understood that although the buttons 70, 72 are illustrated as electrical push buttons, other types of buttons can be used, such as mechanical push buttons, switches and dials. For example, a rocker switch can be used, with different portions of the rocker switch accessible through the different apertures 40, 42. The buttons 70, 72 are accessible because a user can press the corresponding flexible button portions 50, 52 to thereby activate (or deactivate) the underlying buttons. It is also understood that the number of buttons can be different for different embodiments or implementations.

The switch mechanism 20 can be removable, in some embodiments. This allows the switch mechanism 20 to be removed before a sterilization procedure, such as autoclave. In addition, the skeleton 30 of the switch mechanism 20 provides enhanced strength to the switch mechanism, thereby increasing its reliability during a surgical process and its ability to withstand multiple sterilization procedures.

The preceding specific embodiment is illustrative of the practice of the invention. It is to be understood that other expedients known to those skilled in the art or disclosed herein, may be employed without departing from the invention or the scope of the appended claims. For example, the present invention is not limited to surgical instruments employing a cutting element, but may find further applications in which high speed rotation of a relatively small motor is required.

What is claimed is:

1. A surgical instrument comprising an outer casing, a button for controlling an operation of the surgical instrument, and a switch cover for engaging with the outer casing and for providing external access to activate and/or deactivate the button.

2. The surgical instrument of claim 1 wherein the switch cover includes a rigid skeleton and a flexible cover.

3. The surgical instrument of claim 2 wherein the rigid skeleton is made of injection molded plastic.

4. The surgical instrument of claim 3 wherein the rigid skeleton includes an aperture corresponding with the button.

5. The surgical instrument of claim 2 wherein the casing includes a first portion with an indention and the switch cover includes a second portion for releasably and frictionally engaging with the first portion.

6. The surgical instrument of claim 2 wherein the casing includes a first portion with an indention and the switch cover includes a second portion for mechanically engaging with the first portion.

7. The surgical instrument of claim 2 wherein the flexible cover is made of silicon.

8. A surgical instrument comprising:
an outer casing including a first portion for surrounding a motor, a second portion having for housing a plurality of buttons, and a third portion for providing a small body by which a user can control and manipulate the surgical instrument;
a plurality of buttons provided in the second portion of the outer casing, the buttons for controlling an operation of the motor; and
a switch cover for engaging with the second portion of the outer casing, the switch cover including a relatively stiff skeleton and a plurality of relatively flexible portions that correspond with the plurality of buttons when the switch cover is engaged with the second portion of the outer casing, the flexible portions being adapted to provide external control of the buttons while the switch cover is engaged.

9. The surgical instrument of claim 8 wherein the first, second, and third portions of the outer casing are arranged in a stair-step configuration such than the second portion is smaller in diameter than the first portion and the third portion is smaller in diameter than the second portion.

10. The surgical instrument of claim 8 wherein the stiff skeleton is made of injection molded plastic.

11. The surgical instrument of claim 8 wherein the plurality of relatively flexible portions are formed from a single silicon-based membrane formed around the stiff skeleton.

12. The surgical instrument of claim 8 wherein the switch cover includes a side portions for frictionally engage with an indented side portions of the outer casing.

13. The surgical instrument of claim 12 wherein the indented side portions are configured to produce a recessed area inside the second portion.

14. The surgical instrument of claim 13 wherein the recessed area includes at least one protrusion for fitting and securing with corresponding indentions in the switch cover.

15. The surgical instrument of claim 13 wherein the side portions of the switch cover are flared so that they mechanically engage with the recessed area.

16. The surgical instrument of claim 8 wherein at least one of the plurality of buttons is a push-button.

17. A surgical instrument comprising:
an outer casing including a first portion for surrounding a motor, a second portion having for housing a plurality of buttons, and a third portion for providing a small body by which a user can control and manipulate the surgical instrument, wherein the first, second, and third portions of the outer casing are arranged in a stair-step configuration such than the second portion is smaller in diameter than the first portion and the third portion is smaller in diameter than the second portion;
two buttons provided in the second portion of the outer casing, the buttons for controlling an operation of the motor;
a battery for providing power to the motor;
a tool connected to the motor for performing a surgical operation on a human patient; and
a removable switch cover for engaging with the second portion of the outer casing, the switch cover including a relatively stiff skeleton made of injection molded plastic and two relatively flexible portions formed from a single silicon-based membrane formed around the stiff skeleton, wherein the two relatively flexible portions correspond with the two buttons when the switch cover is engaged with the second portion of the outer casing, the flexible portions being adapted to provide external control of the buttons while the switch cover is engaged.

18. The surgical instrument of claim 8 wherein the switch cover includes a side portions for frictionally engage with an indented side portions of the outer casing.

19. The surgical instrument of claim 13 wherein the side portions of the switch cover are flared so that they mechanically engage with the recessed area.
